(19) United States
(12) Patent Application Publication
(10) Pub. No.: US 2017/0143753 A1
(43) Pub. Date: May 25, 2017

(54) SOPHOROLIPID ESTER CHAIN LENGTH DEPENDENCE ON THE INHIBITION OF HUMAN PATHOGENS

(71) Applicants: Richard A. Gross, Plainview, NY (US); Filbert Totsingan, Troy, NY (US)

(72) Inventors: Richard A. Gross, Plainview, NY (US); Filbert Totsingan, Troy, NY (US)

(73) Assignee: Syntehzyme LLC, Rensselaer, NY (US)

(21) Appl. No.: 15/356,552

(22) Filed: Nov. 19, 2016

Related U.S. Application Data
(63) Continuation-in-part of application No. 13/644,563, filed on Oct. 4, 2012, now abandoned.
(60) Provisional application No. 61/543,122, filed on Oct. 4, 2011.

(51) Int. Cl. A61K 31/7084

(52) U.S. Cl. CPC A61K 31/7084

(57) ABSTRACT

A method for inhibitory and bactericidal activities against human pathogens by n-alkyl chain length of modified sophorolipid n-alkyl esters, and preferred n-alkyl chain lengths to enhance sophorolipid n-alkyl ester inhibitory and bactericidal activities against human pathogens.

### STATEMENT OF RELATED APPLICATIONS

This application claims the benefit of and priority on U.S. patent application Ser. No. 13/644,563 having a filing date of 4 Oct. 2012, which claims the benefit of and priority on U.S. Provisional Patent Application No. 61/543,122 having a filing date of 4 Oct. 2011.

### BACKGROUND OF THE INVENTION

1. Technical Field

The present invention relates generally to the field of sophorolipids (SLs) and more specifically to new compositions of matter for uses of sophorolipid alkyl esters as antibacterial agents against human pathogens.

2. Prior Art

Nature has evolved a class of compounds known as sophorolipids. These compounds are classified as a microbial surfactant as they are produced by fermentation of microbes. Fermentations to produce sophorolipids generally use carbohydrate and lipids as carbon sources. Sophorolipids lower surface and interfacial tension and form micelles at and above their critical micelle concentration (CMC). The larger families of microbial biosurfactants have amphiphilic structures; possess a hydrophilic moiety such as a peptide, sugar or oligosaccharide; and possess a hydrophobic moiety including saturated or unsaturated lipid or fatty acids. Sophorolipids are a class of glycolipid biosurfactant (FIG. 1) produced by yeasts, such as Candida bombicola, Yarrowi lipolytica, Candida apicola, and Candida bogoriensis.

Sophorolipids consist of a hydrophilic carbohydrate head group (sophorose) and a hydrophobic fatty acid tail. Sophorose is an unusual disaccharide that consists of two glucose molecules linked β-1,2. Furthermore, sophorose in sophorolipids can be acetylated at the 6′- and/or 6″-positions (FIG. 1). One fatty acid hydroxylated at the terminal (ω-) or subterminal (ω-1) positions is β-glycosidically linked to the sophorose molecule. The fatty acid carboxylic acid group is either free (acidic or open form) or internally esterified generally at the 4″-position (lactonic form, LSL) (FIG. 1 and Table 1). The hydroxyl fatty acid component of sophorolipids generally has 16 or 18 carbon atoms with generally one unsaturated bond. However, the sophorolipid fatty acid can also be further unsaturated (e.g. consisting of ω-1 hydroxylated oleic, linoleic and ω-linoleic acids). Similarly, sophorolipid hydroxyl fatty acids can consist of chain lengths from C12 to C22 having various degrees and positions of unsaturation. As such, sophorolipids synthesized by C. bombicola generally consist of a mixture of molecules that are related. Differences between these molecules are found based on the fatty acid structure (degree of unsaturation, chain length, position(s) of unsaturation and position of hydroxylation), whether they are produced in the lactonic (LSL) or ring-opened form, and the acetylation pattern. The invention described herein is applicable to sophorolipids with all of the above variations in structure.

Work has been carried out to “tailor” sophorolipid (SL) structure during in vivo formation. These studies have mainly involved the selective feeding of different lipophilic substrates. For example, changing the co-substrate from sunflower to canola oil resulted in a large increase (50% to 73%) of the LSL portion of SLs. Also, unsaturated C-18 fatty acids such as oleic acid may be transferred unchanged into sophorolipids. Finally, LSL and acidic sophorolipids are synthesized in vivo from stearic acid with similar yields to oleic acid-derived sophorolipids. Thus, to date, physiological variables during fermentations have provided routes to the variation of sophorolipid compositions.

As noted above, fermentation by different microorganisms, Candida bombicola, Yarrowi alipolytica, Candida apicola, and Candida bogoriensis, leads to sophorolipids of different structure noted above, the variations in sophorolipids based on fatty acid feedstocks and organisms leads to a wide array of sophorolipids including LSL and acidic structures. An additional modification that is relevant to acidic sophorolipids is cleavage of the sophorose moiety to the corresponding glucose-based glucolipids. Treatment of acidic sophorolipids with enzymes that include β-glucuronidase (from Helix pomatia), cellulase (from Penicillium funiculosum), Clara diastase (a mixture of enzymes including amylase, cellulase, peptidase, phosphatase, and sulphatase), galactomannanase (from Aspergillus niger), hemicellulase (from Aspergillus niger), hesperidinase (from Aspergillus niger), inulinase (from Aspergillus niger), pectolyase (from Aspergillus japonicus), or naringinase (from Penicillium decumbens) afford glucolipids over a range of pH values. The antimicrobial activity of glucolipids can also be improved by the invention described herein

The chain length of the fatty acid carbon source fed can result in changes in the predominant fatty acid incorporated into the sophorolipid produced. For example, it has been found that when hexadecane and octadecane were fed in fermentations, over 70% of the hydroxylated fatty acids found in sophorolipids were hexadecanoic and octadecanoic acids, respectively. When shorter alkanes such as tetradecane were fed as substrates in fermentations, only a minor fraction of the sophorolipids produced by the organism consist of the corresponding hydroxylated shorter chain fatty acid. Instead, the vast majority of these shorter chain fatty acids are elongated to either C16 or C18 fatty acids. Similarly, when longer alkanes such as eicosane (C20) are fed to the sophorolipid producing organism, generally longer chain fatty acids are metabolized via β-oxidation to shorter chain length hydroxylated C16 and C18 fatty acids.

Furthermore, the degree of lactonization of sophorolipids and acetylation of the sophorose polar head may be influenced by the carbon source fed during sophorolipid production. For example, it has been found that sophorolipids derived from rapeseed, sunflower and palm oils rich in C18:0 and C18:1 fatty acids are formed with higher levels of diacetylated lactones than sophorolipids produced from the corresponding fatty acid ester feedstocks.

Recombinant sophorolipid producing strains have been reported that allow further control of sophorolipid structure. For example, In order to redirect unconventional substrates towards sophorolipid synthesis, the β-oxidation pathway was blocked on the genome level by knocking out the multifunctional enzyme type 2 (MFE-2) gene. (see FEMS Yeast Res 9 (2009) 610-617). Using this approach, sophorolipids can be produced that are enriched in a hydroxylated alkane, hydroxylated alkene, or hydroxyl fatty acid. Recently, the enzyme responsible for the lactonization of SLs was discovered. The discovery of the gene encoding this lactone esterase (sble) enabled the development of S. bombicola strains that produce either solely lactonic or solely acidic SLs. (see Roelants, S. L. et al., Towards the industrialization of new biosurfactants: Biotechnological opportunities for the lactone esterase gene from Starmerella bombicola, Biotechnology and Bioengineering (2016), 113(3), 550-559. and WO 2013092421 A1). Also, Rhodotorula bogoriensis produces sophorolipids (SLs) that contain 13-hydroxydocosanoic acid (OH—C22) as the lipid moiety (see Solaiman, D. K. Y., et al., Biotechnol. Prog., 31:867-874, 2015). In addition, a recombinant Candida bombicola strain with an acetyltransferase gene knockout can be used to produce sophorolipids without acetylation (see WO 2012080116 A1). The invention disclosed herein can be used to enhance the antimicrobial activity of the above described sophorolipids may they be produced by a recombinant organisms or by a strain that naturally produces sophorolipids that consist of fatty acids that are hydroxylated at unusual positions.

It is known that the n-alkyl chain length of SL-esters affects its interfacial properties at air-water. Modifications of SLs were performed so that the chain length of the n-alkyl group (methyl, ethyl, propyl, butyl, pentyl, and hexyl and related branched alkyl groups) esterified to the sophorolipid fatty acid was varied. The structure and abbreviation used for the corresponding series of SLs is given in Table 1. The effect of the n-alkyl ester chain length on interfacial properties of corresponding sophorolipid analogues was studied. The cmc and minimum surface tension have an inverse relationship with the alkyl ester chain length. That is, cmc decreased to ½ per additional CH₂ group for the methyl, ethyl, and propyl series of chain lengths. These results were confirmed by fluorescence spectroscopy. Adsorption of sophorolipid alkyl esters on hydrophilic solids was also studied to explore the type of lateral associations. These surfactants were found to absorb on alumina but much less on silica. This adsorption behavior on hydrophilic solids is similar to that of sugar-based nonionic surfactants and unlike that of nonionic ethoxylated surfactants. Hydrogen bonding is proposed to be the primary driving force for adsorption of sophorolipids on alumina. Increase in the n-alkyl ester chain length of SLs caused a shift of the adsorption isotherms to lower concentrations. The magnitude of the shift corresponds to the change in cmc of these surfactants.

It has been shown that modified sophorolipids have antibacterial, antifungal, antiviral, and anti-inflammatory properties. In one example, sophorolipids were shown to down-regulate expression of pro-inflammatory cytokines including interleukin. Furthermore, it was found that the antibacterial and antifungal activity of sophorolipids against plant pathogens can be increased relative to the natural SL mixture by modifications such as esterification of fatty acid carboxyl groups and selective acetylation of disaccharide hydroxyl groups.

Existing data suggests that sophorolipids may be useful as antimicrobial agents, antifungal agents, in the treatment of sepsis and septic shock, as virucidal and spermicidal agents, and as antifungal agents. However, there are no examples in the patent or chemical literature that direct one skilled in the art that there is a preferred n-alkyl ester chain length that will lead towards important improvements in antimicrobial activity of SL-esters against human pathogens. See U.S. Pat. No. 7,772,193; U.S. Pat. No. 7,262,178; U.S. patent application Ser. No. 11/020,683; U.S. patent application Ser. No. 12/360,486; and U.S. patent application Ser. No. 13/644,563.

It has been reported that a natural mixture of SLs (non-chemically modified) is active against bacterial pathogens Bacillus subtilis and Propionibacterium acne. Total inhibition of B. subtilis and Propionibacterium acne was observed at the minimum inhibition concentration (MIC) of 50 μg/mL and 150 μg/mL, respectively. Thus, natural sophorolipids may be economically produced but have relative low activity against human pathogen strains. A further serious limitation is that natural lactonic SL has very low solubility in aqueous systems. Composites consisting of microbial polyesters and natural SLs were prepared and were found to have antimicrobial activity against Propionibacterium acnes. Increasing SL concentrations in poly-3-hydroxybutyrate (PHB) and PHB-co-10%-3-hydroxyhexanoate (PHB/HHx) resulted in noticeably improved (PHB/HHx was best) antimicrobial activity based on the size of the zones of inhibition. This invention also can be used to improve the antimicrobial activity of biopolyesters composites by substituting natural SLs with SL-esters having an optimal n-alkyl ester chain length that is disclosed in this invention.

Sophorolipids with C12 and C14 hydroxylfatty acids that were produced from coconut oil are reported to reduce the growth of both gram-positive (S. aureus) and gram-negative (E. coli) in a concentration-dependent manner. In addition, SLs produced using glucose as the hydrophilic source and lauryl alcohol as the hydrophobic source were effective in causing complete cell death of both gram-positive (S. aureus and B. subtilis) and gram-negative (E. coli and P. aeruginosa) in 2-4 hours at concentrations of 1-30 μg/mL. The invention disclosed herein also can be used to improve the antimicrobial activity of SLs with various chain length hydrophobic groups that include C12, C14 fatty acids modification of these structures forming an SL-ester having an optimal n-alkyl ester chain length that is disclosed in this invention.

Quaternary ammonium-terminated C9 peracetylated and unacetylated SL derivatives were found to be effective against gram-positive bacteria with MIC for the most active compounds in the range of 5-10 μg/mL. They exhibited no activity against gram-negative strains. Furthermore, the method used for modification is chemically intense involving multiple steps that would result in products that could not compete based on cost-performance.

Sophorolipids with longer lipid chains (e.g. C22-SL) produced by R. bogoriensis using 13-hydroxydocosanoic acid (OH—C22) were also tested against Propionibacterium acne. Reported data suggest that C22-SLs are able to inhibit the bacterial cells more efficiently than C18-SLs according to zone inhibition assay. As above, the invention disclosed herein also can be used to improve the antimicrobial activity of SLs with longer chain hydroxyl fatty acids such as C22 by converting these molecules to n-alkyl esters with optimal chain length as is disclosed herein.

The SL-ester derivatives disclosed herein widely expand the range of use of sophorolipids by the amelioration of the effects of natural SLs for use in various forms (in composites, on surfaces, in various formulations) to inhibit the growth and kill important human pathogens.

### BRIEF SUMMARY OF THE INVENTION

Briefly stated, the present invention describes sophorolipid ester derivatives for use in killing or inhibiting the growth of human pathogenic bacteria. Sophorolipids derivatives disclosed herein are described based on the predominant fatty acid constituent, 17-hydroxyoleic acid, produced by C. bombicola when fed crude oleic acid as its fatty acid source. However, as changes in the lipid feed (canola oil and rapeseed oil) lead to different sophorolipids as described above, variations in feedstock also will result in changes in the molecular structure of SL-esters that are disclosed herein.

SL-esters disclosed herein include those having the following structures:

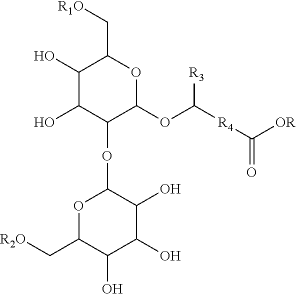

where R₁ and/or R₂ is selected from the following groups: hydrogen and acetyl;
R₃ can be a hydrogen or alkyl group (e.g. methyl);
R₄ is an alkyl chain that normally has between 9 and 19 carbons and normally has unsaturation (C═C bond) at one or more sites; and
R is an alkyl group,
wherein, SL derivatives comprise a family of esters (RO[C═O]) at the SL fatty acid carboxylic acid group that can have the following other structural characteristics: i) may be selectively acylated at sophorose primary hydroxyl groups (C6′ and/or C6″) to form modified sophorolipids with R₁—O and/or R₂—O esters; and ii) have R₄ groups with 0, 1, 2 or 3 double bonds (C═C).

Thus, sophorolipid derivatives disclosed herein were discovered to be highly effective against important human pathogens. Furthermore, this work discloses preferred lengths of the sophorolipid ester R-groups that lead to enhanced antimicrobial activity against human pathogens. This dependence of antimicrobial activity on the length of R groups in RO[C═O] is unexpected and provides an important method to obtain modified sophorolipids with enhanced antimicrobial activity.

### BRIEF DESCRIPTION OF THE FIGURES

FIG. 1 illustrates a structure of open chain (acidic) forms of sophorolipid mixture produced by Candida bombicola.

FIG. 2 illustrates sophorolipid ester derivatives of the open chain form with increasing length.

FIG. 3 illustrates time-kill study of sophorolipid ester derivatives.

### DETAILED DESCRIPTION OF PREFERRED EMBODIMENTS

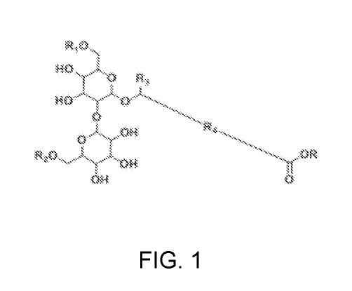

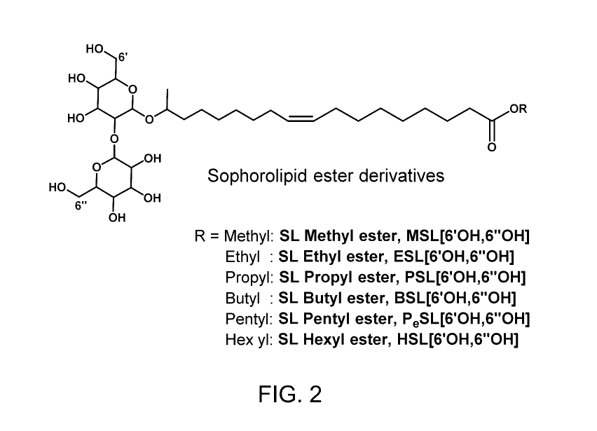

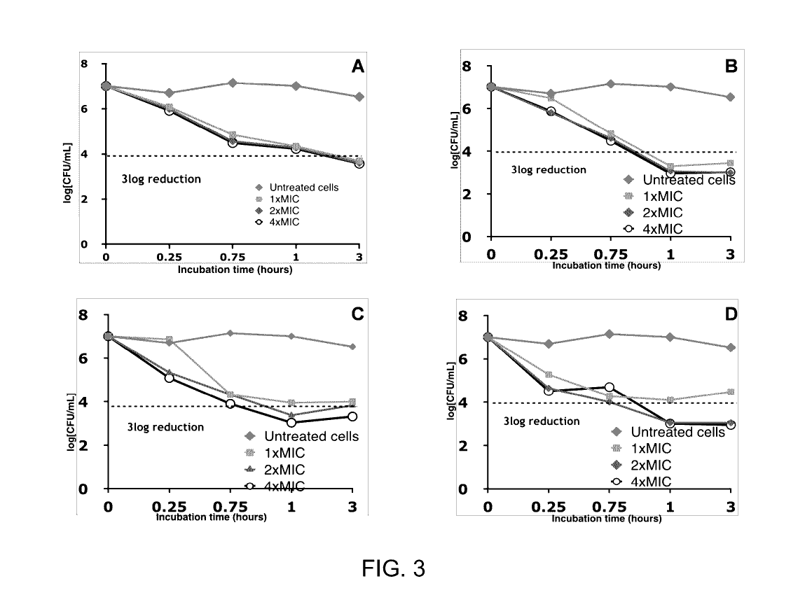

Embodiments of this invention are based on the unexpected discovery that there are preferred lengths of sophorolipid ester R-groups (RO[C═O], FIGS. 1 and 2) that lead to enhanced antimicrobial activity against human pathogens.

The present invention also incorporates additional variations in sophorolipid structures beyond those disclosed herein that do not depart from the scope and spirit of the invention.

The bio-based and modified sophorolipids that comprise the present invention are obtained from fermentations that use as carbon sources pure fatty acids, fatty acid mixtures, pure fatty acid esters, mixtures of fatty acid esters, triglycerides along with carbohydrate sources such as corn syrup, dextrins and glucose using a fermentation process comprising a wild-type or engineered yeast strain such as Candida bombicola. These sophorolipids generally consist of a hydrophilic carbohydrate head, sophorose, and a hydroxylated fatty acid tail with 16 or 18 carbon atoms with saturation and unsaturation. Sophorose is an unusual disaccharide that consists of two glucose molecules linked β-1,2. Furthermore, sophorose in sophorolipids can be acetylated on the 6′- and/or 6″-positions (FIG. 1).

The hydrophobic fatty acid tail of sophorolipids normally is hydroxylated at the terminal or subterminal positions and is 3-glycosidically linked to the sophorose molecule (the polar head group). The fatty acid carboxylic acid group is either free (acidic or open form; FIG. 1) or internally esterified generally at the 4″-position (lactonic form, LSL). The hydroxy fatty acid component of sophorolipids generally has 16 or 18 carbon atoms where C18 chains generally have one unsaturated bond. However, the sophorolipid fatty acid can also be fully saturated. As such, sophorolipids synthesized by C. bombicola consist of a mixture of molecules that are related. Differences between these molecules are found based on the fatty acid structure (degree of unsaturation, chain length, and position of hydroxylation), existing in the lactonic or ring-opened form, and the acetylation pattern. Sophorolipids derivatives disclosed herein are described based on the predominant fatty acid constituent, 17-hydroxyoleic acid, produced by C. bombicola when fed crude oleic acid or rapeseed oil or canola oil as its fatty acid source. However, sophorolipid derivatives disclosed herein can be produced by using sophorolipids prepared from a wide range of other fatty acid and carbohydrate feedstocks by a fermentation process. Furthermore, natural sophorolipids used in this invention can be produced by a range of sophorolipid producing microbes such as Candida bombicola, Starmerella clade, Rhodotorula bogoriensis, and Wickerhamiella domericqiae. Sophorolipid compositions derived from other fatty acid sources that have different chain length, position of hydroxylation that forms a glycosidic bond with the sophorose polar head group and the degree of unsaturation are included within the invention disclosed in this specification without departing from the scope and spirit of the invention.

Synthesis of SL Methyl Ester

In a round-bottom flask equipped with a reflux condenser, dry diacetyl lactonic sophorolipid (29 mmol) was dissolved in dry methanol (50 mL). To this mixture, was slowly added 10 mL of sodium methoxide prepared separately by adding a small piece of Na metal (170 mg, 0.25 eq) in dry methanol. The resulting mixture was refluxed under N₂ for 2 h and the reaction was monitored by TLC [CHCl₃/CH₃OH (8:2), staining solution: Cerium ammonium molybdate (CAM)]. Upon completion, the reaction mixture was cooled to room temperature, acidified with glacial acetic acid to pH 4-5 and concentrated by rotoevaporation. Precipitation in ice-cold water, followed by filtration and dryness under vacuum yielded the desired product as an off-white solid (91%).

Synthesis of SL Ethyl Ester

In a round-bottom flask equipped with a reflux condenser, dry diacetyl lactonic sophorolipid (29 mmol) was dissolved in dry ethanol (50 mL). To this mixture, was slowly added 10 mL of sodium ethoxide prepared separately by adding a small piece of Na metal (170 mg, 0.25 eq) in dry ethanol. The resulting mixture was refluxed under N₂ for 3 h and the reaction was monitored by TLC [CHCl₃/CH₃OH (8:2), staining solution: Cerium ammonium molybdate (CAM)]. Upon completion, the reaction mixture was cooled to room temperature, acidified with glacial acetic acid to pH 4-5 and concentrated by rotoevaporation. Precipitation in ice-cold water, followed by filtration and dryness under vacuum yielded the desired product as an off-white solid (87%).

Synthesis of SL Propyl Ester

In a round-bottom flask equipped with a reflux condenser, dry diacetyl lactonic sophorolipid (29 mmol) was dissolved in dry 1-propanol (50 mL). To this mixture, was slowly added 10 mL of sodium propoxide prepared separately by adding a small piece of Na metal (170 mg, 0.25 eq) in dry 1-propanol. The resulting mixture was stirred at 80° C. under N₂ for 3 h and the reaction was monitored by TLC [CHCl₃/CH₃OH (8:2), staining solution: Cerium ammonium molybdate (CAM)]. Upon completion, the reaction mixture was cooled to room temperature, acidified with glacial acetic acid to pH 4-5 and concentrated by rotoevaporation. Precipitation in ice-cold water, followed by filtration and dryness under vacuum yielded the desired product as an off-white solid (81%).

Synthesis of SL Butyl Ester

In a round-bottom flask, dry diacetyl lactonic sophorolipid (14.5 mmol) was dissolved in dry n-butanol (25 mL). To this mixture, was slowly added 5 mL of sodium butoxide prepared separately by adding a small piece of Na metal (80 mg, 0.24 eq) in dry n-butanol. The resulting mixture was stirred at 80° C. under N₂ for 3 h and the reaction was monitored by TLC [CHCl₃/CH₃OH (8:2), staining solution: Cerium ammonium molybdate (CAM)]. Upon completion, the reaction mixture was cooled to room temperature, acidified with glacial acetic acid to pH 4-5 and excess of n-butanol was removed by rotoevaporation. The solid residue was re-dissolved in a minimum volume of ethanol (10 mL) and Precipitation in ice-cold water (500 mL), followed by filtration and dryness under vacuum yielded the desired product as a light solid (80%).

Synthesis of SL Pentyl Ester

In a round-bottom flask, dry diacetyl lactonic sophorolipid (14.5 mmol) was dissolved in dry 1-pentanol (25 mL). To this mixture, was slowly added 5 mL of sodium pentoxide prepared separately by adding a small piece of Na metal (80 mg, 0.24 eq) in dry 1-pentanol. The resulting mixture was stirred at 80° C. under N₂ for 3-4 h and the reaction was monitored by TLC [CHCl₃/CH₃OH (8:2), staining solution: Cerium ammonium molybdate (CAM)]. Upon completion, the reaction mixture was cooled to room temperature, acidified with glacial acetic acid to pH 4-5 and excess of 1-pentanol was removed by rotoevaporation. The solid residue was re-dissolved in a minimum volume of ethanol (10 mL) and Precipitation in ice-cold water (500 mL), followed by filtration and dryness under vacuum led to the desired product as an off-white solid (78%).

Synthesis of SL Hexyl Ester

In a round-bottom flask, dry diacetyl lactonic sophorolipid (14.5 mmol) was dissolved in dry 1-hexanol (25 mL). To this mixture, was slowly added 5 mL of sodium hexoxide prepared separately by adding a small piece of Na metal (80 mg, 0.24 eq) in dry 1-hexanol. The resulting mixture was stirred at 80° C. under N₂ for 3-4 h and the reaction was monitored by TLC [CHCl₃/CH₃OH (8:2), staining solution: Cerium ammonium molybdate (CAM)]. Upon completion, the reaction mixture was cooled to room temperature, acidified with glacial acetic acid to pH 4-5 and excess of 1-hexanol was removed by rotoevaporation. The solid residue was re-dissolved in a minimum volume of ethanol (10 mL) and Precipitation in ice-cold water (500 mL), followed by filtration and dryness under vacuum led to the desired product as an off-white solid (77%). Alternatively, flash chromatography was used for purification of the crude product.

Results and Discussion

One class of sophorolipid derivatives are sophorolipid esters (RO[C═O]) that are formed by esterification at the SL fatty acid carboxylic acid group (FIG. 2). Esterification of sophorolipids is generally achieved by alcoholysis of natural sophorolipid mixtures. Esters of varying chain lengths are included in this invention. Scheme 1 depicts the synthetic pathway used to prepare these SL derivatives (Scheme 1, reaction 2). Other methods can be used to prepare SL-esters such as lipase-catalyzed esterification of various alkanols directly to the fatty acids of ring-opened SL or by enzymatic ring-opening of LSL by an alkanol.

A second class of sophorolipid derivatives is the natural mixture produced from fermentation (Scheme 1, reaction 2), containing di-acetylated lactonic sophorolipids as the main component.

Table 1 shows representative examples of antimicrobial sophorolipid derivatives claimed in the present invention, as well as their corresponding molecular weights (MW).

TABLE 1
Sophorolipid derivatives and pure sophorolipid components of the natural mixture were used in bacterial human
pathogen assays. The hydroxylated fatty acid of the natural mixture is predominantly 17-hydroxyoleic
acid. However, other fatty acid constituents with variations in chain length and unsaturation may also be present.

| SL derivative | Name | MW |
|---|---|---|
| 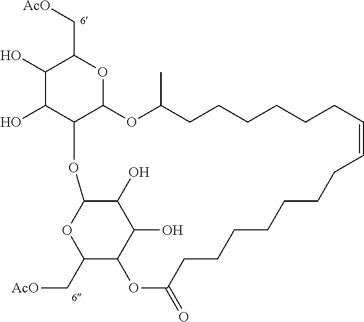 [SMILES: CC(=O)OCC1OC2OC(C)CCCCCC/C=C\CCCCCCCC(=O)OC3C(COC(C)=O)OC(OC2C(O)C1O)C(O)C3O] | LSL 6′,6″-diacetate (natural component) | 688.8 |
| 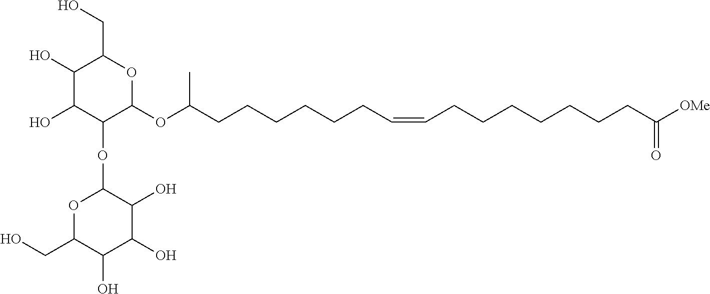 [SMILES: COC(=O)CCCCCCC/C=C\CCCCCCC(C)OC1OC(CO)C(O)C(O)C1OC1OC(CO)C(O)C(O)C1O] | SL Methyl ester | 636.8 |
| 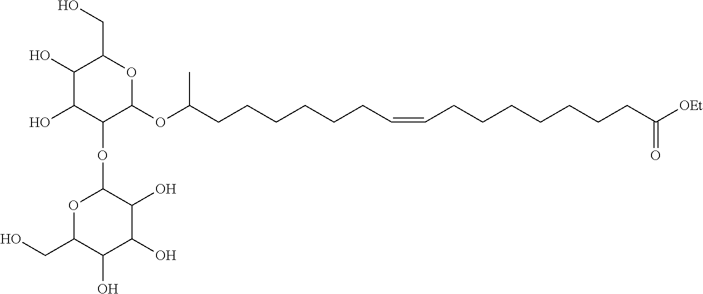 [SMILES: CCOC(=O)CCCCCCC/C=C\CCCCCCC(C)OC1OC(CO)C(O)C(O)C1OC1OC(CO)C(O)C(O)C1O] | SL Ethyl ester | 650.8 |
| 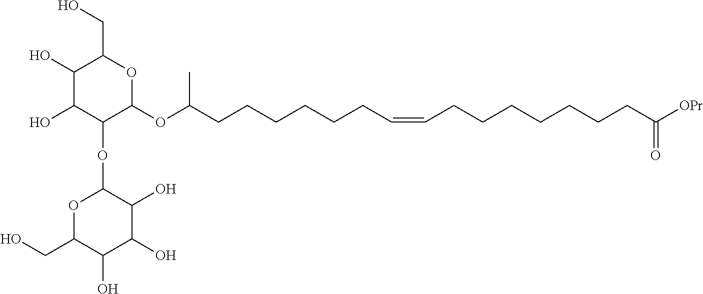 | SL Propyl ester | 664.8 |
| 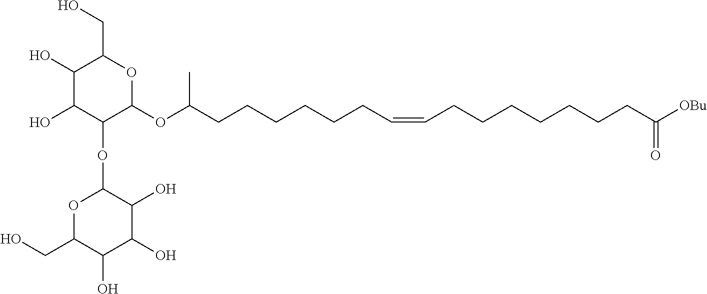 | SL Butyl ester | 678.9 |
| 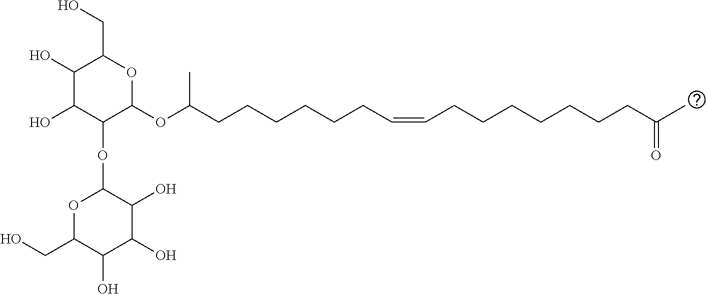 [SMILES: CC(=O)CCCCCCC/C=C\CCCCCCC(C)OC1OC(CO)C(O)C(O)C1OC1OC(CO)C(O)C(O)C1O] | SL Pentyl ester | 692.9 |
| 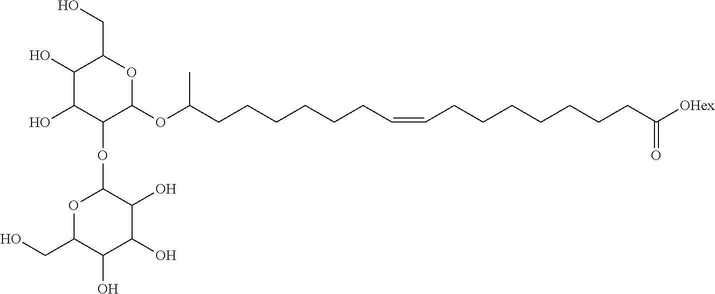 | SL Hexyl ester | 706.9 |

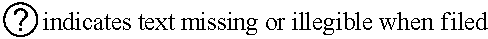

Representative Examples of Inhibitory Activity Against Bacterial Human Pathogens

### Example 1: Minimum Inhibitory Concentration (MIC) of Diacetate Lactonic SL, SL Methyl Ester, SL Ethyl Ester, SL Propyl Ester, SL Pentyl Ester and SL Hexyl Ester

Sophorolipid ester derivatives, as well as the diacetate lactonic sophorolipid have inhibitory activity against a range of human pathogenic bacteria, which was confirmed by experiments and observations. Sophorolipid samples were dissolved in pure DMSO to a final concentration of 2.56 mg/mL that was used as a stock solution. Except the first row, the wells of a 96-well microplate were loaded with 100 μL of sterilized PBS buffer. The stock solution (20 μL) and sterilized PBS buffer pH 7.4 (180 μL) was added into the first row of the 96-well microplate and serially diluted (2-fold dilution) from 256 μg/mL to 2 μg/mL using PBS buffer. After serial dilution, 100 μL of fresh bacteria cells (10⁶ cfu/mL) suspended in Soy broth (SB) or Mueller Hinton broth (MHB) culture medium were added to each well and the plates were incubated for 16 or 30 hours. Wells with only cells in 5% DMSO in PBS/medium (no SL) and with only 5% DMSO in PBS/medium (no SL and no cells) were used as positive and negative controls, respectively. The minimum inhibitory concentration (MIC), defined as the lower SL concentration that shows no visible growth of bacteria cells, was determined to measure antimicrobial activity of sophorolipid-derived compounds. Lactonic SL as well as all SL esters except SL hexyl ester (no activity against all strains) and SL pentyl ester (no activity against B. Subtilis) were active against all three gram-positive bacterial strains tested (B. Cereus, B. Subtilis, Listeria innocua). The unexpected result in Table 2 is that, as the chain length of the sophorolipid ester increased from methyl to ethyl, propyl and butyl, the MIC values for B. Cereus, B. Subtilis and L. Innocua decrease. The pentyl ester has similarly low MIC values as the butyl ester derivative for B. Cereus and L. Innocua. However, for the pentyl ester, the MIC against B. Subtilis increases to >128 μg/mL. Furthermore, for B. Cereus, B. Subtilis and L. Innocua, SL propyl, butyl and pentyl esters have antimicrobial activity that is higher or comparable to that of natural diacetylated lactonic sophorolipid. However, the low water solubility of diacetylated lactonic SL limits its use in aqueous formulations.

TABLE 2
Minimum Inhibitory Concentration (MIC) of lactonic
sophorolipid diacetate and sophorolipid ester derivatives.

| | Minimum Inhibitory Concentration (MIC), μg/mL | | |
|---|---|---|---|
| Derivative | B. Cereus¹ | B. Subtilis¹ | Listeria Innocua² |
| Lactonic SL³ | 16 | 8 | 32 |
| SL Methyl ester | 32 | 32 | 64 |
| SL Ethyl ester | 32 | 32 | 32 |
| SL Propyl ester | 16 | 25 | 25 |
| SL Butyl ester | 8 | 8 | 8 |
| SL Pentyl ester | 8 | >128 | 8 |
| SL Hexyl ester | >128 | >128 | >128 |

¹Incubation time: 16 h
²Incubation time: 30 h
³Natural SL used to prepare SL esters

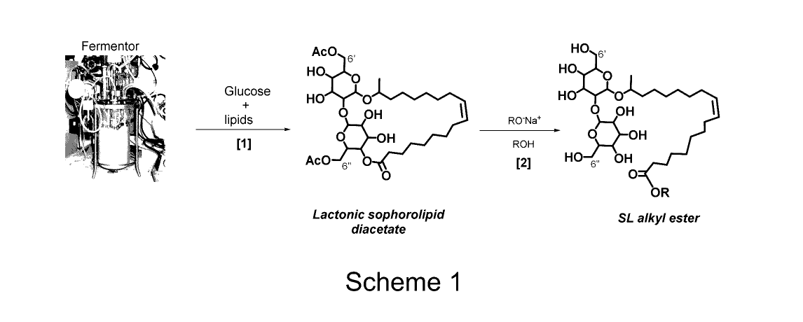

Bactericidal activity (time-kill study): an in vitro assay was performed to test the bactericidal effect of each sophorolipid derivatives on human pathogen bacteria viability. Time-dependent killing of pathogens by sophorolipid derivatives was evaluated by exposing roughly 10⁷ cfu/mL of bacterial cells to 1×MIC, 2×MIC and 4×MIC of lactonic SL and SL-ester derivatives in PBS buffer pH 7.4. After 15 min, 45 min, 1 h and 3 h incubation, cells were spread on agar plates, incubated at 37° C. for 18 hours and examined for colonies. An untreated cells vial (i.e. no SL added) was used as the control. The results are reported in FIG. 3.

Sophorolipid derivatives used in this invention showed useful inhibitory activity against bacterial human pathogens. To further investigate if the growth inhibition by sophorolipid derivatives is associated with cell death, i.e. if sophorolipid derivatives are bactericidal or bacteriostatic, time-kill studies were performed. Time-dependent killing studies (FIG. 3) showed that at 2×MIC and 4×MIC, the SL esters (methyl; propyl; and butyl) killed more than 99.9% of cells (3 log reduction) within 1 hour. Moreover, the killing rate was higher for these sophorolipid ester derivatives than natural di-acetylated lactonic sophorolipid that requires almost 2 hours to reach a 3 log reduction.

A summary of synthetic pathways developed to prepare the sophorolipid derivatives used in the present invention is shown in Scheme 1, which shows the synthesis of lactonic sophorolipid diacetate (by fermentation) and sophorolipid alkyl esters (by transesterification/alcoholysis).

The above detailed description of the embodiments, and the examples, are for illustrative purposes only and are not intended to limit the scope and spirit of the invention, and its equivalents, as defined by the appended claims. One of ordinary skill in the art will recognize that many variations can be made to the invention disclosed in this specification without departing from the scope and spirit of the invention.

### REFERENCES

Felse, P. A., et al., Sophorolipid biosynthesis by Candida bombicola from industrial fatty acid residues, Enzyme and Microbial Technology, 40 (2), 316-323 (2007).
Zhang, L. et al., Synthesis and interfacial properties of sophorolipid derivatives, Colloids and Surfaces A: Physicochem. Eng. Aspects; 240; 75-82 (2004).
Singh, S. K., et al., Regioselective Enzyme-Catalyzed Synthesis of Sophorolipid Esters, Amides and Multifunctional Monomers, J. Org. Chem.; 68; 5466-5477 (2003).
Guilmanov, V., et al., Oxygen Transfer Rate and Sophorose Lipid Production by Candida bombicola, Biotechnol. and Bioeng.; 77(5), 489-494 (2002).
Bisht, K., et al., Enzyme-Mediated Regioselective Acylations of Sophorolipids, J. Org. Chem., 64:3, 780-789 (1999).
Marchal, R., et al., Method of production of sophorosides by fermentation with fed batch supply of fatty acid esters or oils, U.S. Pat. No. 5,616,479, 1997.
Trummler, K., et al., An integrated microbial/enzymatic process for production of rhamnolipids and L-(+)-rhamnose from rapeseed oil with Pseudomonas sp. DSM 2874, Eur. J. Lipid Sci. Technol.; 105, 563-571 (2003).
Monteiro, L. M., et al., Development and characterization of a new oral dapsone nanoemulsion system: permeability and in silico bioavailability studies, Int. J. Nanomedicine.; 7; 5175-5182 (2012).
Cutter, C. N., et al., Improved antimicrobial activity of nisin-incorporated polymer films by formulation change and addition of food grade chelator, Let. Appl. Microbiol.; 33; 325-328 (2001)
Kim, K., et al., Characteristics of Sophorolipid as an antimicrobial agent, J. Microbiol. Biotechnol.; 12(2), 235-241 (2002)
Pulate, V. D., et al., Antimicrobial and SEM Studies of Sophorolipids Synthesized using Lauryl Alcohol, J. Surfact. Deterg.; 17, 543-552 (2014).
Morya, V. K., et al., Production and characterization of low molecular weight sophorolipid under fed-batch culture, Bioresource Technol.; 143, 282-288 (2013).
Delbeke, E. I. P., et al., A new class of antimicrobial biosurfactants: quaternary ammonium sophorolipids, Green Chem.; 17, 3373-3377 (2015).
Solaiman, D. K. Y., et al., High-Titer Production and Strong Antimicrobial Activity of Sophorolipids from Rhodotorula bogoriensis, Biotechnol. Prog.; 31(4), 867-874.
Ashby, R. D., et al., Biopolymer scaffolds for use in delivering antimicrobial sophorolipids to the acne-causing bacterium propionibacterium acnes, New Biotechnonoly; 28(1) (2011).
German Patent Application No. 10 2009 045 077 A1
International Patent Publication No. WO 2004 044 216 A1
International Patent Publication No. WO 2006 069 175 A2
International Patent Publication No. WO 2011 127 101 A1
International Patent Publication No. WO 2012 080 116 A1

1. A method for inhibiting human pathogenic bacteria comprising:
providing a modified sophorolipid ester derivative,
wherein the modified sophorolipid ester derivative is obtained through transesterification/alcoholysis of a natural sophorolipid.

2. The method as claimed in claim 1, wherein the modified sophorolipid derivative is obtained from at least one of a pure natural sophorolipid mixture, a crude natural sophorolipid, and a mixture directly collected from fermentation of culture broth.

3. The method as claimed in claim 1, wherein the modified sophorolipid derivative is synthesized from a natural sophorolipid produced by fermentation of a sophorolipid producing strain using lipid feedstocks selected from the group consisting of oleic acid, high oleic acid oils, canola oil, rapeseed oil, vegetable oil, fatty acid, fatty acid ester, and alkane.

4. The method as claimed in claim 1, wherein the modified sophorolipid derivative is synthesized from sophorolipid producing strains selected from the group consisting of Candida bombicola, Yarrowi alipolytica, Candida apicola, and Candida bogoriensis, Starmerella bombicola, Starmerella clade, Rhodotorula bogoriensis, and Wickerhamiella domericqiae.

5. The method as claimed in claim 4, wherein at least one of the following is present:
the modified sophorolipid derivative is synthesized from a recombinant sophorolipid producing strain that has been genetically modified to improve fermentation production efficiency;
the β-oxidation pathway is blocked on the genome level,
elevated expression of the lactone esterase that enable the production of sophorolipids with very high lactonic contents,
elevated expression levels of gene encoding this lactone esterase (sble) enabled the development of S. bombicola strains that produce either solely lactonic or solely acidic SLs,
elevated expression levels or deletion of the acetyltransferase gene and alterations in the glucose transferase enzymes.

6. The method as claimed in claim 1, wherein the modified sophorolipid ester derivative is selected from those that have 2, 3, 4 or 5-carbons.

7. The method as claimed in claim 6, wherein the modified sophorolipid ester derivative has 4 carbons.

8. The method as claimed in claim 7, wherein the modified sophorolipid ester derivative is sophorolipid butyl ester.

9. The method as claimed in claim 1, wherein the modified sophorolipid ester derivative is used in combination with other active or inert ingredients used in antibacterial formulations that have a wide variety of physical forms selected from the group consisting of liquids, pastes, gels, powders, granules, semisolids, colloidal materials, composites, fibers, and coatings.

10. The method as claimed in claim 1, wherein the modified sophorolipid ester derivative has the formula: 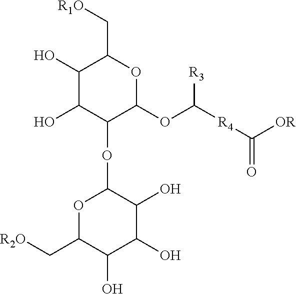
where R₁ and/or R₂ is hydrogen or acetyl;
R₃ is a hydrogen or alkyl group;
R₄ is an alkyl chain that normally has between 9 and 19 carbons and normally has unsaturation (C═C bond) at one or more sites; and
R is an alkyl group
wherein, the sophorolipid derivatives comprise a family of esters (RO[C═O]) at the SL fatty acid carboxylic acid group that has at least one of the following other structural characteristics (i) may be selectively acylated at sophorose primary hydroxyl groups (C6′ and/or C6″) to form modified sophorolipids with R₁—O and/or R₂—O esters; and (ii) have R₄ groups with 0, 1, 2 or 3 double bonds (C═C).

11. A bactericide for human pathogens comprising a modified sophorolipid ester derivative obtained through transesterification/alcoholysis of a natural sophorolipid.

12. The bactericide as claimed in claim 11, wherein the modified sophorolipid derivative is obtained from at least one of a pure natural sophorolipid mixture, a crude natural sophorolipid, and a mixture directly collected from fermentation of culture broth.

13. The bactericide as claimed in claim 11, wherein the modified sophorolipid derivative is synthesized from a natural sophorolipid produced by fermentation of a sophorolipid producing strain using lipid feedstocks selected from the group consisting of oleic acid, high oleic acid oils, canola oil, rapeseed oil, vegetable oil, fatty acid, fatty acid ester, and alkane.

14. The bactericide as claimed in claim 11, wherein the modified sophorolipid derivative is synthesized from sophorolipid producing strains selected from the group consisting of Candida bombicola, Yarrowi alipolytica, Candida apicola, and Candida bogoriensis, Starmerella bombicola, Starmerella clade, Rhodotorula bogoriensis, and Wickerhamiella domericqiae.

15. The bactericide as claimed in claim 14, wherein at least one of the following is present:
the modified sophorolipid derivative is synthesized from a recombinant sophorolipid producing strain that has been genetically modified to improve fermentation production efficiency;
the β-oxidation pathway is blocked on the genome level,
elevated expression of the lactone esterase that enable the production of sophorolipids with very high lactonic contents,
elevated expression levels of gene encoding this lactone esterase (sble) enabled the development of S. bombicola strains that produce either solely lactonic or solely acidic SLs,
elevated expression levels or deletion of the acetyltransferase gene and alterations in the glucose transferase enzymes.

16. The bactericide as claimed in claim 11, wherein the modified sophorolipid ester derivative is selected from those that have 2, 3, 4 or 5-carbons.

17. The bactericide as claimed in claim 16, wherein the modified sophorolipid ester derivative has 4 carbons.

18. The bactericide as claimed in claim 17, wherein the modified sophorolipid ester derivative is sophorolipid butyl ester.

19. The bactericide as claimed in claim 11, wherein the modified sophorolipid ester derivative is used in combination with other active or inert ingredients used in antibacterial formulations that have a wide variety of physical forms selected from the group consisting of liquids, pastes, gels, powders, granules, semisolids, colloidal materials, composites, fibers, and coatings.

20. The bactericide as claimed in claim 11, wherein the modified sophorolipid ester derivative has the formula: 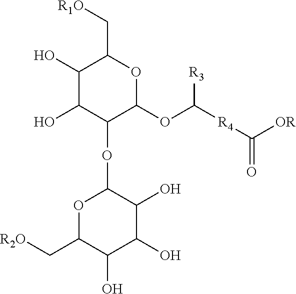
where R₁ and/or R₂ is hydrogen or acetyl;
R₃ is a hydrogen or alkyl group;
R₄ is an alkyl chain that normally has between 9 and 19 carbons and normally has unsaturation (C═C bond) at one or more sites; and
R is an alkyl group
wherein, the sophorolipid derivatives comprise a family of esters (RO[C═O]) at the SL fatty acid carboxylic acid group that has at least one of the following other structural characteristics (i) may be selectively acylated at sophorose primary hydroxyl groups (C6′ and/or C6″) to form modified sophorolipids with R₁—O and/or R₂—O esters; and (ii) have R₄ groups with 0, 1, 2 or 3 double bonds (C═C).
